(19) United States
(12) Patent Application Publication
(10) Pub. No.: US 2006/0079783 A1
(43) Pub. Date: Apr. 13, 2006

(54) Method and system for deriving a fetal heart rate without the use of an electrocardiogram in non-3D imaging applications

(76) Inventor: David W. Clark, Windham, NH (US)

Correspondence Address: PHILIPS INTELLECTUAL PROPERTY & STANDARDS, P.O. BOX 3001, BRIARCLIFF MANOR, NY, 10510, US

(21) Appl. No.: 11/222,151

(22) Filed: Sep. 8, 2005

Related U.S. Application Data
(60) Provisional application No. 60/614,722, filed on Sep. 30, 2004.

(51) Int. Cl. A61B 8/02

(52) U.S. Cl. 600/450

(57) ABSTRACT

A system and method are provided for determining a fetal heart rate from ultrasound imaging data in near real-time. The heart rate is determined by analyzing spatial points on ultrasound cardiac volumes and calculating the peak spectral frequency of the changes in ultrasonic characteristics of the spatial points.

### BACKGROUND OF THE INVENTION

The present invention relates generally to medical devices. More particularly, the present invention relates to a method and device for deriving a fetal heart rate without the use of an electrocardiogram in non-3D imaging applications.

Medical ultrasound imaging has become a popular means for visualizing and medically diagnosing the condition and health of interior regions of the human body. With this technique, an acoustic transducer probe, which is attached to an ultrasound system console via an interconnection cable, is held against the patient's tissue by the sonographer where it emits and receives focused ultrasound waves in a scanning fashion. The scanned ultrasound waves, or ultrasound beams, allow the systematic creation of image slices of the patient's internal tissues for display on the ultrasound console. The technique is generally quick, painless, fairly inexpensive and safe, even for such uses as fetal imaging.

Ultrasound imaging systems commonly in use generate and transmit ultrasound signals to map internal tissue typography, vascular fluid flow rates, and abnormalities. The systems typically incorporate several methods, or modes, of imaging, i.e. Brightness Mode (B-Mode), Harmonic, Spectral Doppler, and Color Flow.

Each imaging method has its characteristic uses and limitations. B-Mode imaging is typically used to image the structure of internal tissue and organs with high spatial resolution. Generally to achieve this degree of spatial resolution, short-duration ultrasound pulses are advantageous. Harmonic imaging uses the harmonic frequencies produced from nonlinear wave propagation. Harmonic imaging can reduce clutter, sidelobes, and aberration compared to the more traditional fundamental B-mode imaging, but typically involves compromising spatial resolution.

Color Flow imaging is primarily used to image blood flow and locate abnormal or turbulent flows within the cardiovascular system. Color Flow images are usually overlaid on to a B-Mode structural image. However, the ultrasound properties necessary for proper Color Flow imaging differ from those used in B-Mode. Color Flow imaging requires multiple pulses to detect motion, and longer duration ultrasound pulses than commonly used for B-Mode scans for sensitivity. Low ultrasound pulse repetition rates are desirable for slow-flowing veins, but for the faster flows found in the arteries and heart, higher ultrasound pulse repetition rates are necessary to properly avoid aliasing errors.

Spectral Doppler uses a very large number of ultrasound pulses (or a continuous wave) in the same direction, and converts the resulting echo data stream into a frequency spectrum versus time display and an audio output. Spectral Doppler provides more detailed blood flow dynamics information for one location, in contrast to Color Flow's simple estimation for many locations. Typically Color Flow is used to decide where to place the Spectral Doppler sample location.

3D ultrasound imaging involves scanning the ultrasound pulses over a volume rather than one plane, either mechanically or electrically. Typically the volume is scanned as a series of 2D planes. The echo data is usually displayed either as a volume-rendered image or as 2D planar images sliced through the volume data. The name 4D imaging is sometimes used when 3D volumes are acquired and/or displayed rapidly enough to see motion of the structure being imaged (time being the fourth dimension).

The heart and the fetus have been the two main applications for 3D ultrasound imaging, because both involve significant volumes of liquid that are nearly transparent to ultrasound, so the anatomy can be visualized relatively easily in three dimensions. Particularly with color flow imaging, a 3D acquisition is typically too slow for the fast motion of the heart, so for adult or pediatric 3D cardiac exams an electrocardiogram is used to synchronize the ultrasound acquisition over multiple cardiac cycles. An electrocardiogram is impractical for a fetal heart exam, however. Nelson, Sklansky, and Pretorius at the University of California at San Diego published a technique for deriving the fetal heart rate from the 2D B-mode images in a slow (many heart cycle) 3D scan through the fetal heart, then using the derived heart rate to shuffle the 2D images into 3D volumes of the fetal heart at multiple points of the cardiac cycle. That technique (called the NSP technique hereafter) is implemented in several commercially available ultrasound systems, and will be described in more detail below. The NSP technique has only been applied to post-processing 3D image acquisitions of the fetal heart, and it depends on having significant cardiac motion in the 2D B-mode images.

An objective of the present disclosure is to extend the NSP technique to derive, display or use the heart rate without needing an electrocardiogram, in situations other than 3D imaging of the fetal heart. A further objective of the present disclosure is to extend the NSP technique to operate on data other than 2D B-mode slices of a 3D acquisition, which is particularly useful in non-cardiac exams where there is very little cardiac-cycle motion in the B-mode images and electrocardiograms are seldom used. A further objective of the present disclosure is to extend the NSP technique to operate repetitively, including using overlapping time segments, to provide rapidly updated heart rate estimates in a live imaging situation.

### SUMMARY OF THE INVENTION

An embodiment of the present disclosure provides a method for determining a heart rate from a set of ultrasound images. A set of ultrasound images is acquired at a predetermined time sampling rate. A series of spatial points are then selected and mapped on each image of the set of ultrasound images. The data corresponding to each of said spatial points is processed to determine a spectral peak frequency. Finally, the spectral peak frequency is scaled according to the predetermined time sampling rate, giving the heart rate.

An additional embodiment of the present disclosure provides for an ultrasound medical imaging system. The ultrasound medical imaging system includes an ultrasound imager having an ultrasound transducer, a processor, and a video display.

### BRIEF DESCRIPTION OF THE DRAWINGS

These and other features, aspects, and advantages of the present invention will become better understood with regard to the following description, appended claims, and accompanying drawings wherein:

FIG. 1 is a flow chart illustrating the steps for determining a heart rate from a set of ultrasound images in accordance with an embodiment of the present disclosure; and

FIG. 2 is a schematic view illustrating an ultrasound imaging system in accordance with an embodiment of the present disclosure.

### DESCRIPTION OF THE PREFERRED EMBODIMENTS

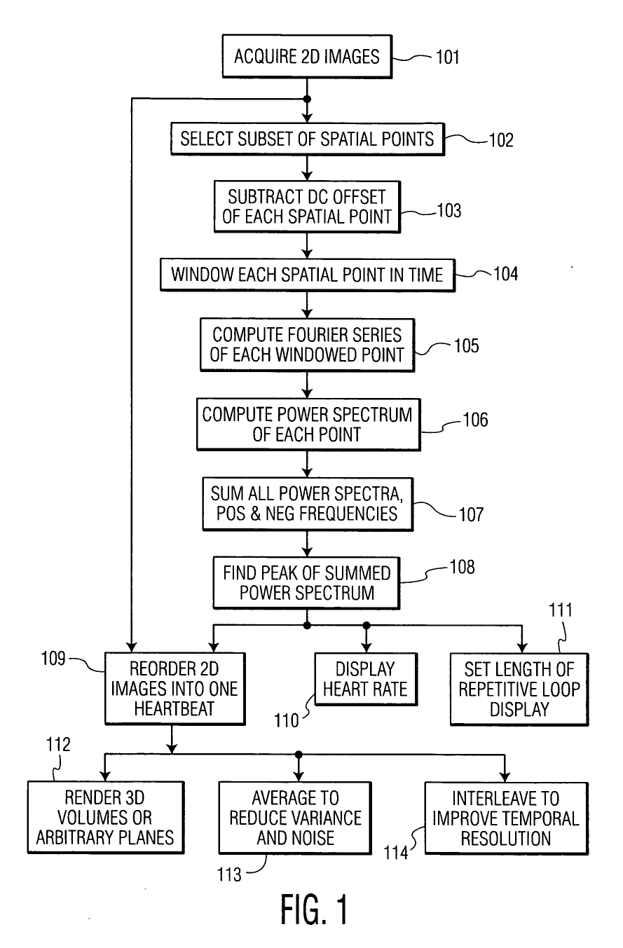

As shown in FIG. 1, an embodiment of the present disclosure provides steps for determining a heart rate from a set of ultrasound cardiac images. In step 101, an ultrasound imaging device begins a scan to acquire 2D cardiac images (later it will be described how this can be other than 2D cardiac images). Proceeding on, step 102 selects a subset of spatial points, such as a uniformly spaced grid. The selection step may be performed either manually or as an automated process. In step 103, the DC offset is subtracted from each spatial point. Proceeding to step 104, the spatial points selected in step 102 are plotted with respect to time and a window function is applied to the data in step 105. Two appropriate window functions are the Hann and Hamming functions. A power spectrum is calculated in step 106 for the windowed data. In step 107, all the power spectra are summed, including both positive and negative frequencies. From the summed power spectra, a power spectrum peak is derived and processed along with the time sampling rate between image scans to determine the heart rate in step 108. The summed power spectrum covers the frequency range from zero to half the sample rate (the sample rate is the 2D frame rate). The location of the peak of the power spectrum is therefore at some fraction of the sample rate. Multiplying that by the sample rate in Hertz gives the heart rate in Hertz, and multiplying by 60 gives the heart rate in beats per minute.

There are several alternative ways that the derived heart rate may be used, as shown in FIG. 1. The prior art uses the heart rate to rearrange slowly elevation-swept 2D images of a fetal heart into multiple 3D volumes at different times of the heart cycle, step 109. Other alternative uses of the derived heart rate, shown in FIG. 1, are a subject of the present disclosure. The length of a repetitive loop display of some subset of the 2D images (typically called a cine-loop) can be set to a whole number of cardiac cycles to minimize discontinuities when the loop wraps from end back to beginning, step 111.

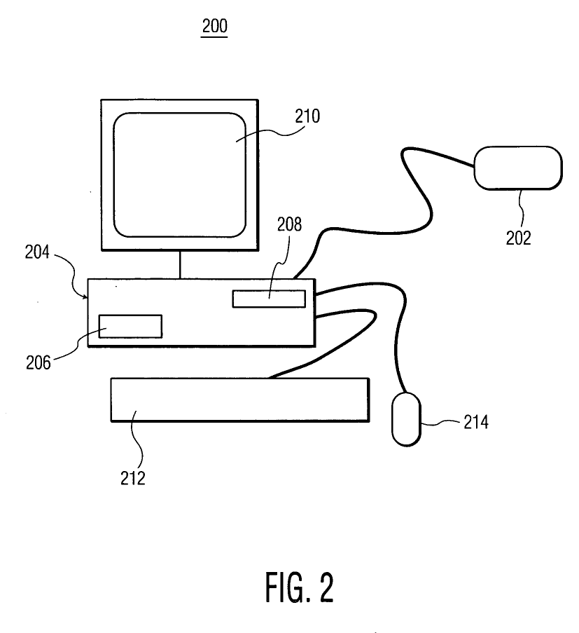

Another embodiment of the present disclosure, shown in FIG. 2, provides for a medical ultrasound imaging system 200. The system 200 includes an ultrasound transducer assembly 202 connected to an imaging workstation 204. The imaging workstation 204 contains one or more processors 206 and at least one storage device 208, such as a hard drive, RAM disk, etc. The storage device(s) 208 may be used for storing the controlling and imaging software for the ultrasound system 200 as well as providing temporary and long term storage of image data acquired by the ultrasound transducer 202. The ultrasound imaging system 200 also provides a video display 210 and user input devices, including a keyboard 212 and a mouse 214.

The processor 206 is configured to execute the controlling and imaging software. The imaging software allows the operator of the system 200 to visualize and manipulate the data received from the ultrasound transducer 202. Additionally, the imaging software includes subroutines to perform the method of the present disclosure as exemplified by FIG. I and described in detail above.

The described embodiments of the present invention are intended to be illustrative rather than restrictive, and are not intended to represent every embodiment of the present invention. Various modifications and variations can be made without departing from the spirit or scope of the invention as set forth in the following claims both literally and in equivalents recognized in law.

1. A method for determining a heart rate from a set of ultrasound images comprising the steps of:
acquiring a set of ultrasound images at a predetermined time sampling rate;
selecting spatial points, and mapping said spatial points on each image of said set of ultrasound images;
processing data corresponding to each of said spatial points, said processing step being performed substantially simultaneous with said acquiring step;
determining a spectral peak frequency for said power spectrum; and
scaling said spectral peak frequency according to said predetermined time sampling rate, said scaled spectral peak frequency is said heart rate.

2. The method of claim 1, wherein the processing step further comprises the steps of:
acquiring data for each of said spatial points;
applying an apodization function to said data; and
calculating a power spectrum for said apodized data.

3. The method of claim 1, wherein said selecting step is performed upon said acquisition of a first ultrasound image of said set of ultrasound images.

4. A system for determining a heart rate from a set of ultrasound images comprising:
means for acquiring a set of ultrasound images at a predetermined time sampling rate;
means for selecting spatial points, and mapping said spatial points on each image of said set of ultrasound images;
means for processing data corresponding to each of said spatial points;
means for determining a spectral peak frequency for said power spectrum; and
means for scaling said spectral peak frequency according to said predetermined time sampling rate, said scaled spectral peak frequency is said heart rate.

5. The system of claim 4, wherein the means for processing comprises:
means for acquiring data for each of said spatial points;
means for applying an apodization function to said data; and
means for calculating a power spectrum for said apodized data.

6. The system of claim 4, wherein said means for selecting is activated upon acquisition of a first ultrasound image of said set of ultrasound images.

7. The system of claim 4, wherein said data processing is performed substantially simultaneous with said ultrasound image acquisition.

8. The system of claim 4, wherein said selection of spatial points is performed upon a first ultrasound image of said acquired set of ultrasound images.
